(19) United States
(12) Patent Application Publication
(10) Pub. No.: US 2021/0299032 A1
(43) Pub. Date: Sep. 30, 2021

(54) IMPROVING THE QUALITY OF FORMULATION CLARITY & SENSORY WITH AN EXTRACT COMPRISING A XANTHONE ANTIOXIDANT

(71) Applicant: L'OREAL, Paris (FR)

(72) Inventors: Brian Scott BODNAR, Manasquan, NJ (US); Jennifer HAGHPANAH, Newtown, CT (US)

(73) Assignee: L'OREAL, Paris (FR)

(21) Appl. No.: 16/835,822

(22) Filed: Mar. 31, 2020

(51) Int. Cl. A61K 8/9789; A61K 8/49; A61K 8/34; A61K 8/44; A61K 8/73; A61K 8/42; A61Q 19/00

(52) U.S. Cl. CPC A61K 8/9789; A61K 8/4973; A61K 8/345; A61K 8/34; A61Q 19/007; A61K 8/731; A61K 8/4953; A61K 8/4926; A61K 8/42; A61K 8/442

(57) ABSTRACT

A cosmetic composition and a method of preparing a cosmetic composition that includes at least one xanthonoid; at least about 10 wt. % of Vitamin C; at least one hydrotrope from about 0.8 to about 1.5 wt. %.; a solvent comprising water, at least one glycol and ethyl alcohol; pantothenic acid; hydroxypropyl methylcellulose; and wherein the cosmetic composition is stable, translucent and lubricated.

### FIELD OF THE INVENTION

The present invention is generally directed to a cosmetic composition and a method for preparing the cosmetic composition that includes a xanthone antioxidant and wherein the composition is translucent and has a very appealing sensory aspect when applied to the skin

### BACKGROUND OF THE INVENTION

The formation of free radicals is a widely accepted pivotal mechanism leading to skin aging. Free radicals are highly reactive molecules with unpaired electrons that can directly damage various cellular structures, including membranes, lipids, proteins, RNA and DNA. The damaging effects of these reactive oxygen species are induced internally during normal metabolism and externally through various oxidative stresses. UV exposure and environmental pollution can accelerate skin aging by producing free radicals in skin. Antioxidants protect cells from the damage of oxidative stress by scavenging free radicals and inhibiting the following oxidation reactions. The topical application of antioxidants is broadly used in skin care products to prevent skin aging; however, antioxidants are generally molecules which are difficult to dissolve in cosmetic formulations. Moreover, once dissolved, the nature of the chemical structure of antioxidants promotes crystallization in the cosmetic medium and in cosmetic formulations, making the production and storage of cosmetic formulations containing antioxidants a major challenge.

For the foregoing reasons, it is an object of the present invention to provide compositions containing a xanthonoid, and vitamin C, in which it is critical to control the amount of hydrotropes, pantothenic acid and pantothenic acid derivatives, thickeners, and amount of glycols and alcohols in the solvent system.

### BRIEF SUMMARY OF THE DISCLOSURE

The present disclosure relates to cosmetic compositions that are unique in comparison to conventional cosmetic compositions because they contain and deliver a high level of Vitamin C in an antioxidant serum, which has optical translucency, prevents crystallization of actives (hereby contained and referenced within the term “stability”), and maintains a satisfactory sensory aspect when applied on skin (i.e. a good lubricity).

In the instant disclosure, it was found that while a wide variety of acceptable mixtures of most ingredients are able to provide optical translucency and prevent crystallization of actives without Hydroxypropyl methylcellulose (HPMC), the lubricity and general sensory aspects of the cosmetic composition when applied on skin was not appealing. When HPMC was added to thicken the serum for sensorial reasons, applicants unexpectedly found that control of caffeine, solvent, pantothenic acid and pantothenic acid derivatives, carboxylic acid-containing antioxidants, and solvent was critical for optimal translucency and stability. While not wishing to be bound by any particular theory, the applicants believe that in cosmetic compositions containing vitamin C, xanthonoid, and HPMC, the ratio of hydrotropes, pantothenic acid and pantothenic acid derivatives, carboxylic acid-containing antioxidants, and glycols are critical to control optical translucency and prevent crystallization. The cosmetic compositions typically include:
a. At least one xanthonoid;
b. At least about 10% of Vitamin C;
c. At least one hydrotrope from about 0.8 to about 1.5% wt.;
d. A solvent comprising water and, at least one glycol and ethyl alcohol;
e. Pantothenic acid, derivatives of pantothenic acid, and mixtures thereof;
f. Hydroxypropyl methylcellulose, ethylcellulose, methyl cellulose, and mixtures thereof;
g. At least one carboxylic acid-containing antioxidant; and
wherein the cosmetic composition is stable, translucent and provides a lubricated feel when applied on skin; and
wherein all weight percentages are based on the total weight of the cosmetic composition.

In one or more embodiments, the at least one xanthonoid may be chosen from Mangifera indica leaf extract, tomentonone, zeyloxanthonone, calozeyloxanthone, morellic acid, forbesione methylswertianin, bellidifolin, mangostin, normangostin, gartanin, euxanthic acid, and mixtures thereof.

In one or more embodiments, the at least one xanthanoid is present from about 0.1 to about 1.0 wt. % based on the total weight of the cosmetic composition.

In some embodiments, the composition comprises at least one hydrotrope that comprises caffeine, hydroxy urea, and combinations thereof. In one or more embodiments, the at least one hydrotrope is present from about 0.8 to about 1.2 wt. % based on the total weight of the cosmetic composition.

In some embodiments, the solvent comprises water, glycols chosen from pentylene glycol, propylene glycol, dipropylene glycol, and combinations thereof; and ethyl alcohol. In one or more embodiments, the glycols are present from about 15 to about 25 wt. % based on the total weight of the cosmetic composition.

In some embodiments, pantothenic acid, derivatives of pantothenic acid, and mixtures thereof are present from about 1.5 to about 2.5 wt. % based on the total weight of the cosmetic composition. In some embodiments, pantothenic acid, derivatives of pantothenic acid, and mixtures thereof is panthenol.

In some embodiments, the panthenol is present from about 1.5 to about 2.5 wt. % based on the total weight of the cosmetic composition.

In some embodiments, Hydroxypropyl methylcellulose; ethylcellulose, methyl cellulose, and mixtures thereof are present from about 0.1 to about 1.5 wt. % based on the total weight of the cosmetic composition.

In one or more embodiments, the Hydroxypropyl Methylcellulose is present from about 0.1 to 1.5 wt. % based on the total weight of the cosmetic composition.

In some embodiments, the presence of Hydroxypropyl methylcellulose requires control of the ratio between the xanthonoid, vitamin C, the hydrotrope, the glycols, the ethyl alcohol, and the panthenol, pantothenic acid, and/or pantothenic acid derivative in order to obtain an aesthetically pleasing formulation with translucency, stability, and lubricity when applied to the skin.

Another aspect of the instant disclosure can include:
a. From about 0.1 to about 1.0 wt. % of at least one xanthonoid chosen from Mangifera indica leaf extract, tomentonone, zeyloxanthonone, calozeyloxanthone, morellic acid, forbesione methylswertianin, bellidifolin, mangostin, normangostin, gartanin, euxanthic acid, and mixtures thereof;
b. From at least about 10 wt. % of Vitamin C;
c. From about 0.8 to about 1.5 wt. % of at least one hydrotrope chosen from caffeine, urea, nicotinic acid, and esters, salts, or amides thereof, and combinations thereof;
d. From about 18 to about 22 wt. % of a solvent comprising water and at least one glycol chosen from pentylene glycol, propylene glycol, dipropylene glycol and combinations thereof;
e. From about 10 to about 20 wt. % of ethyl alcohol;
f. From about 1.0 to about 2.0% of panthenol;
g. From about 0.2 to about 1.0 wt. % of Hydroxypropyl Methylcellulose;
h. from about 0.5 to about 5 wt. % of at least one carboxylic acid-containing antioxidant;
   wherein the cosmetic composition is stable, translucent and provides a lubricated feel when applied to skin;
   wherein all weight percentages are based on the total weight of the cosmetic composition.

In another exemplary embodiment, a method for treating skin comprising applying a cosmetic composition to the skin.

The cosmetic compositions are useful for treating skin, for example, the skin of the face and neck of a human. Thus, the instant disclosure relates to methods of treating the skin comprising application of the cosmetic composition of the instant disclosure to the skin. The cosmetic compositions are additionally useful in methods for treating skin dryness, repairing skin damage due to photoaging, and diminishing the appearance of wrinkles, dark spots, and uneven skin texture. The aforementioned methods may be non-therapeutic.

### DETAILED DESCRIPTION OF THE DISCLOSURE

Where the following terms are used in this specification, they are used as defined below.

The terms “comprising,” “having,” and “including” are used in their open, non-limiting sense.

The terms “a” and “the” are understood to encompass the plural as well as the singular.

The terms “lubricity” or “lubricated feel” are understood to explain a feeling of low friction when gently rubbed, and are understood to be a feeling attributing to the composition when applied to the skin unless otherwise noted.

The term “translucent” is understood to indicate that the formula allows a reasonable amount of light to pass through when stored in a clear container and when placed in front of a light source. Therefore, the resulting “translucent” formula may be completely clear or appear cloudy as described above, but still lets light pass through. Of special note, the term “translucent” does not apply to the color of the formulation stored in a container or when applied to the skin.

The term “stability” herein is understood to describe a cosmetic composition which does not change significantly in appearance following standardized storage protocols, which may include storage of formulations at various temperatures from about 4 degrees Celsius up to about 45 degrees Celsius for prolonged periods of up to 6 months. In particular, “stability” within the present disclosure relates to the visual appearance (either aided by using a microscope or unaided) of crystals or solid particles that precipitate out of the formulation during periods of prolonged, temperature-controlled storage.

The compositions and methods of the instant disclosure can comprise, consist of, or consist essentially of the essential elements and limitations of the disclosure described herein, as well as any additional or optional ingredients, components, or limitations described herein or otherwise useful.

All percentages, parts and ratios herein are based upon the total weight of the compositions of the instant disclosure, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Other than in the operating examples, or where otherwise indicated, all numbers expressing quantities of ingredients and/or reaction conditions are to be understood as being modified in all instances by the term “about,” meaning within +/−5% of the indicated number.

As used herein, the expression “at least one” is interchangeable with the expression “one or more” and thus includes individual components as well as mixtures/combinations.

The term “active material” as used herein with respect to the percent amount of an ingredient or raw material, refers to 100% activity of the ingredient or raw material.

“Cosmetically acceptable” means that the item in question is compatible with a keratinous substrate such as skin and hair. For example, a “cosmetically acceptable carrier” means a carrier that is compatible with a keratinous substrate such as skin and hair.

The term, “a mixture thereof”, “mixtures thereof”, or “combinations thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counter-ion. This list of counter-ions, however, is non-limiting.

The term “INCI” is an abbreviation of International Nomenclature of Cosmetic Ingredients, which is a system of names provided by the International Nomenclature Committee of the Personal Care Products Council to describe personal care ingredients.

The term “weight ratio” or “mass ratio” as used herein, references the amount of a substance in proportion to a mixture containing said substance, and is calculated by dividing the amount of said substance by weight contained in the mixture by the weight of the mixture containing said substance. As an example, a weight ratio of 0.4 for substance A in a mixture of A, B, and C indicates that the weight of substance A divided by the total weight of substances A, B, and C is 0.4.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Some of the various categories of components identified may overlap. In such cases where overlap may exist and the composition includes both components (or the composition includes more than two components that overlap), an overlapping compound does not represent more than one component. For example, a fatty acid may be characterized as both a nonionic surfactant and a fatty compound. If a particular composition includes both a nonionic surfactant and a fatty compound, a single fatty acid will serve as only the nonionic surfactant or as only the fatty compound (the single fatty acid does not serve as both the nonionic surfactant and the fatty compound).

All publications and patent applications cited in this specification are herein incorporated by reference, and for any and all purposes, as if each individual publication or patent application were specifically and individually indicated to be incorporated by reference. In the event of an inconsistency between the instant disclosure and any publications or patent application incorporated herein by reference, the instant disclosure controls.

The instant disclosure relates to cosmetic compositions comprising a combination of ingredients within a certain ratio that improve the feeling, aesthetic, and the stability of the compositions. The cosmetic compositions of the instant disclosure, in their broadest sense, is in the form of a serum and typically include:
a. At least one xanthonoid;
b. At least about 10 wt. % of Vitamin C;
c. At least one hydrotrope from about 1.0 to about 1.5 wt. %.;
d. A solvent comprising water, at least one glycol and ethyl alcohol;
e. Pantothenic acid, derivatives of pantothenic acid, and mixtures thereof;
f. Hydroxypropyl methylcellulose, ethylcellulose, methyl cellulose and mixtures thereof;
g. At least one carboxylic acid-containing antioxidant; and
wherein the cosmetic composition is stable, translucent and provides a lubricated feel when applied on skin; and
wherein all weight percentages are based on the total weight of the cosmetic composition.

Suitable components, such as those listed below, may be included or excluded from the formulations for the cosmetic compositions depending on the specific combination of other components, the form of the cosmetic compositions, and/or the use of the formulation (e.g., a lotion, a serum, gel, cream, etc.).

Xanthonoid

In an embodiment of the instant disclosure, the at least one xanthonoid is chosen from Mangifera indica leaf extract, tomentonone, zeyloxanthonone, calozeyloxanthone, morellic acid, forbesione methylswertianin, bellidifolin, mangostin, normangostin, gartanin, euxanthic acid, and mixtures thereof. In some embodiments, the at least one xanthanoid is present from about 0.1 to about 1.0 wt. % based on the total weight of the cosmetic composition.

In an embodiment of the instant disclosure, the xanthonoid is largely insoluble in water.

Thus, the at least one xanthanoid is present from about 0.1, 0.12, 0.14, 0.16, 0.18, 0.20, 0.22, 0.24, 0.26, 0.28, 0.30, 0.32, 0.35, 0.40 to about 0.4, 0.42, 0.44, 0.46, 0.48, 0.50, 0.52, 0.54, 0.56, 0.58, 0.60, 0.62, 0.64, 0.66, 0.68, 0.70, 0.75, 0.80, 0.85, 0.90, 0.95, or 1.0 wt. % based on the total weight of the cosmetic composition.

Ascorbic Acid (Vitamin C)

The cosmetic compositions include ascorbic acid. The ascorbic acid may pure L-ascorbic acid (vitamin C), and is typically present in the cosmetic composition in an amount of at least about 10 wt. % based on the total weight of the composition.

The ascorbic acid may be L-ascorbic acid, a mixture of L-ascorbic acid and R-ascorbic acid, and/or a racemic mixture. The ascorbic acid may also be a derivative of L-ascorbic acid, such as ascorbyl glucoside or ascorbyl palmitate. In one embodiment, the ascorbic acid comprises only L-ascorbic acid, essentially only L-ascorbic acid, and/or at least 75 wt. %, at least 85 wt. %, at least 90 wt. %, at least 95 wt. %, or at least 99 wt. % of L-ascorbic acid based on the total weight of ascorbic acid.

Carboxylic Acid-containing Antioxidants

The cosmetic compositions may also include Carboxylic Acid-containing antioxidants, which contain a carboxylic acid functional group and are able to quench free radicals and other electron-deficient reactive species. Examples of carboxylic acid-containing antioxidants include but are not limited to ferulic acid, salicylic acid, derivatives of ferulic or salicylic acids, and mixtures thereof. In one embodiment, the carboxylic acid-containing antioxidants comprise at least 0.5 wt. % ferulic acid and/or salicylic acid, and/or derivatives and mixtures thereof. In some embodiments, the carboxylic acid-based antioxidants comprise at least 1.0 wt. % ferulic acid and/or salicylic acid, and/or derivatives and mixtures thereof.

Hydrotropes

In accordance with the instant disclosure, the composition includes at least one hydrotrope. In some embodiments, the composition includes two or more hydrotropes. At least one hydrotrope refers to one or a combination of two or more hydrotropes.

Many hydrotropes have aromatic structure with an ionic moiety, while some of them are linear alkyl chains, as listed in the table below. Although hydrotropes noticeably resemble surfactants and have the ability to reduce surface tension, their small hydrophobic units and relatively shorter alkyl chain distinguish them as a separate class of amphiphiles. Consequently, their hydrophobicity is not sufficient enough to create well organized self-associated structures, such as micelles, even with a high concentration.

Common hydrotropic molecules include: sodium 1,3-benzenedisulfonate, sodium benzoate, sodium 4-pyridinecarboxylate, sodium salicylate, sodium benzene sulfonate, caffeine, sodium p-toluene sulfonate, sodium butyl monoglycolsulfate, 4-aminobenzoic acid HCl, sodium cumene sulfonate, N,N-diethyl nicotinamide, N-picolylnicotinamide, N-allylnicotinamide, 2-methacryloyloxyethyl phosphorylcholine, resorcinol, butylurea, pyrogallol, N-picolylacetamide 3.5, procaine HCl, proline HCl, nicotinamide, pyridine, 3-picolylamine, sodium ibuprofen, sodium xylenesulfonate, ethyl carbamate, pyridoxal hydrochloride, sodium benzoate, 2-pyrrolidone, ethylurea, N,N-dimethylacetamide, N-methylacetamide, and isoniazid. Hydrotropes can be found in Lee J. et al., “Hydrotropic Solubilization of Paclitaxel: Analysis of Chemical Structures for Hydrotropic Property”, Pharmaceutical Research, Vol. 20, No. 7, 2003; and Lee S. et al., “Hydrotropic Polymers: Synthesis and Characterization of Polymers Containing Picolylnicotinamide Moieties”, Macromolecules, 36, 2248-2255, 2003.

Cosmetically acceptable hydrotropes refers to hydrotropes that can be used in cosmetic compositions. While hydrotropes represent a broad class of molecules used in various fields, cosmetic applications will be limited due to safety and tolerance restrictions. Suitable hydrotropes for use in cosmetics include, but are not limited to, caffeine, sodium PCA (sodium salt of pyrrolidone carbonic acid), sodium salicylate, urea, and hydroxyethyl urea. The suitability of a hydrotrope for use in cosmetic compositions can be determined using tests known in the art for determining effects on skin, and toxicity to humans. Although these hydrotropes are given as an example, it will be appreciated that other hydrotropes compatible with cosmetic applications known in the art may be used. In some embodiments, the composition comprises at least one hydrotrope chosen from caffeine, hydroxy urea and combinations thereof.

In accordance with the various embodiments, the total amount of hydrotropes present in the composition can range from about 0.8 to about 1.5 wt. % based on the total weight of the cosmetic composition.

In some embodiments, the at least one hydrotrope is present in an amount from about 0.80, 0.82, 0.84, 0.86, 0.88, 90, 0.92, 94, 0.96, 0.98, 1.0, 1.2, 1.22, 1.24, 1.26, 1.28, 1.30 to about 1.30, 1.32, 1.34, 1.36, 1.38, 1.40, 1.42, 1.44, 1.46, 1.48, or 1.5 wt. % based on the total weight of the cosmetic composition.

Solvents

Water

The total amount of water in the cosmetic composition can vary, but is typically about 30 to about 95 wt. %, based on the total weight of the cleansing composition. In some instances, total amount of water is about 30 to about 90 wt. %, about 30 to about 85 wt. %, about 30 to about 80 wt. %, about 35 to about 90 wt. %, about 35 to about 85 wt. %, about 35 to about 80 wt. %, about 40 to about 90 wt. %, about 40 to about 85 wt. %, about 40 to about 80 wt. %, about 45 to about 90 wt. %, about 45 to about 85 wt. %, about 45 to about 80 wt. %, about 50 to about 90 wt. %, about 50 to about 85 wt. %, about 50 to about 80 wt. %, about 55 to about 90 wt. %, about 55 to about 85 wt. %, about 55 to about 80 wt. %, about 60 to about 90 wt. %, about 60 to about 85 wt. %, about 60 to about 80 wt. %, about 65 to about 90 wt. %, about 65 to about 85 wt. %, or about 65 to about 80 wt. %, based on the total weight of the cosmetic composition.

Organic Solvents

Non-limiting examples of organic solvents include, for example, alcohols (for example, C1-15, C1-10, or C1-6 alcohols), organic solvents, polyols (polyhydric alcohols and glycols (e.g., glycerin, butylene glycol, caprylyl glycol, etc.), and a mixture thereof.

Non-limiting examples of organic solvents include monoalcohols and polyols such as ethyl alcohol, isopropyl alcohol, propyl alcohol, benzyl alcohol, and phenylethyl alcohol, or glycerin or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of organic solvents include alkanediols such as 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In some embodiments, the organic solvents can include glycols chosen from pentylene glycol, propylene glycol, dipropylene glycol, and combinations thereof; and ethyl alcohol.

In some various embodiments, the glycols are present from about 15, 15.5, 16, 16.5, 17, 17.5, 18, 18.5, 19, 19.5 to about 19.5, 20, 20.5, 21, 21.5, 22, 22.5, 23, 23.5, 24, 24.5, or 25 wt. % based on the total weight of the cosmetic composition.

The total amount solvent(s) in the cosmetic composition can vary but is typically about 10 to about 80 wt. %, based on the total weight of the cosmetic composition. In some cases, the total amount of solvents is about 10.5 to about 70.5 wt. %, about 15 to about 70.5 wt. %, about 20 to about 0.5 to about 10 wt. %, about 0.5 to about 8 wt. %, about 0.5 to about 6 wt. %, about 0.5 to about 5 wt. %, about 1 wt. %, about 2 wt. %, about 3 wt. %, or about 4 wt. %, including all ranges and subranges there between.

Pantothenic Acids

Panthothenic acids that are present in the cosmetic composition, according to the various embodiments, include, but are not limited to, Panthenol, pantothenic acids, pantothenic acid derivatives, and mixtures thereof. Panthenol itself can be considered a derivative of pantothenic acid (vitamin B5).

In an embodiment, panthenol is present in an amount of about 1.5 to about 2.5 wt. %, or preferably, about 1.5 to 2.0 wt. %, or more preferably, about 1.5 to 1.8 wt. %, based on the total weight of the cosmetic composition, including ranges and sub-ranges there between.

Thus, panthenol is present, by weight, based on the total weight of the composition, in an amount of from about 1.5, 1.6, 1.7, 1.8, 1.9, to about 1.9, 2.0, 2.1, 2.2, 2.3, 2.4, or 2.5 wt. %, including increments and ranges therein and there between.

Polysaccharide Derivatives

Hydroxypropyl Methylcellulose

In accordance with the instant disclosure, the composition includes hydroxypropyl methycellulose.

Polysaccharide Derivatives that are present in the cosmetic composition, according to the various embodiments, include, but are not limited to, hydroxypropyl methylcellulose (HPMC), hydroxypropyl cellulose (HPC), polyvinylpyrrolidone (PVP), ethylcellulose, methylcellulose, or combinations thereof. Although these polysaccharide derivative are given as an example, it will be appreciated that other polysaccharide derivative compatible with cosmetic applications known in the art may be used.

In accordance with the various embodiments, hydroxypropyl methycellulose is present in a given composition in an amount of from about 0.01, 0.02, 0.03, 0.04, 0.05, 0.06, 0.07, 0.08, 0.09, 0.1, 0.15, 0.2, 0.25, to about 0.25, 0.3, 0.35, 0.4, 0.45, 0.5, 0.55, 0.6, 0.65, 0.7, 0.75, 0.8, 0.85, 0.9, 0.95, 1.0, 1.2, 1.4, or 1.5 wt. % based on the total weight of the cosmetic composition.

Optional Components

In some embodiments, there may be one or more actives present in the cosmetic composition, according to the disclosure, the active selected from, for example, humectants, such as acetamide MEA, glycols, such as glycerin and propylene glycol; alcohol; anti-microbial components such as zinc pyrrolidone carboxylic acid (zinc PCA); caprylyl glycol; salicylic acid, alpha hydroxy acid; anti-oxidant compounds, including, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria baicalensis root extract), pine bark extract (Pinus pinaster bark/bud extract), ellagic acid; and vitamins and vitamin derivatives, such as panthenol, tocopherol and other Vitamin E derivatives, ascorbic acid; and combinations thereof.

In some embodiments, there may be one or more other components present in the cosmetic composition, according to the disclosure, the additives selected from, preservatives, such as phenoxyethanol; surfactants, including silicone surfactants, W/O and O/W surfactants; oils, including cosmetic oils, fatty compounds, silicone oils such as dimethicone, botanical oils and essential oils; emulsifiers, including silicone emulsifiers; elastomers; fillers such as clays, talc, organic thickeners with for instance, anionic, cationic, nonionic, and amphoteric polymeric associative thickeners and combinations thereof; carbomers; polymers; penetrants; sequestrants; fragrances; dispersants; film-forming agents; ceramides; opacifiers and combinations thereof.

Although the aforementioned optional components are given as an example, it will be appreciated that other optional components compatible with cosmetic applications known in the art may be used.

In accordance with the various embodiments, amounts of optional components present in the composition can range from about 0 to about 50 weight percent, from about 0.5 to about 30 weight percent, from about 1.5 to about 20 weight percent, and from about 5.0 to about 15 weight percent.

In some embodiments, one or more actives, alone or in combination, each can be present in the composition according to the disclosure from about 0.05 to about 50 weight percent, from about 0.05 to about 2.5 weight percent, from about 0.1 to about 2.0 weight percent, from about 0.25 to about 1.5 weight percent, and from about 0.5 to about 1.25 weight percent.

In some embodiments, one or more other components, such as vitamins, preservatives, and the like, alone or in combination, each can be present in the composition according to the disclosure from about 0.05 to about 50 weight percent, from about 0.05 to about 25 weight percent, from about 0.1 to about 10 weight percent, from about 0.25 to about 5.0 weight percent, and from about 0.5 to about 3.5 weight percent. The following examples are intended to further illustrate the present invention. They are not intended to limit the invention in any way. Unless otherwise indicated, all parts are by weight.

Thus, the one or combination of optional components may be present by weight, based on the total weight of the composition, from about 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 25, 30, 35, 40, 45 to about 50 weight percent, including increments and ranges therein and there between.

The following examples are intended to further illustrate the present invention. They are not intended to limit the invention in any way. Unless otherwise indicated, all parts are by weight.

### EXAMPLES

Measurement of Lubricity: Lubricity was measured in all example formulations by applying a controlled aliquot of formula to the back of the hand or on forearm followed by assessment and scoring of intensity of slip or lubricated feeling during the application from 1 to 5 or through relative scoring (where formulas were compared with one another).

Measurement of Translucency: Translucency was measured in all example formulations by measuring the absorbance by UV/Vis spectroscopy of a 200 microliter aliquot of the example formula in a standard cuvet-based or microtiter plate-based UV spectrophotometer. The absorbance value at 600 nm was used to report translucency, where the higher absorbance values at 600 nm correlated with a lower visual translucency for all samples. Threshold for absorbance values at 600 nm determined as “translucent” or turbid were based on comparison with visual assessment of control samples that were found to be translucent enough to be determined acceptable and samples that were too cloudy and therefore determined to be not translucent enough to be acceptable.

Measurement of stability: Stability of all example formulas was measured by storing samples in temperature-controlled chambers at temperatures ranging from 4 degrees Celsius to 45 degrees Celsius and checking each sample periodically over a 2 to 6 months period of time by visual or microscopic inspection. Example formulas were deemed to be stable if no crystallization was observed after 2 months of storage at all temperatures. In the inventive and comparative examples, the stability and likelihood of samples to show signs of crystallization under the stability protocol described above was irrespective of the amount of sample added to the jar or vial; however, in most cases about 1 fluid ounce (about 30 mL) of sample, or more generally, a range of 15 to 40 mL of sample were tested under the stability protocol at each temperature.

Representative examples of inventive and comparative compositions are shown in Table 1, and Inventive compositions may be prepared according to methods consistent with those described herein.

TABLE 1
Inventive and comparative cosmetic
compositions (components shown as percentages based on
the total weight of the composition)

| | | Comp. | Comp. |
|---|---|---|---|
| | Inv. Ex. 1 | Ex. 2 | Ex. 3 |
| Mangifera | 0.625 | 0.625 | 0.625 |
| Indica (Mango) | | | |
| Leaf Extract/ | | | |
| Mangifera | | | |
| Indica Leaf | | | |
| Extract | | | |
| Vitamin C | 15 | 15 | 15 |
| Hydrotrope | 1.0 | 1.0 | 1.0 |
| Glycols | 19 | 19 | 17 |
| Panthenol | 1.7 | 3 | 1 |
| Hydroxypropyl | 0.4775 | 0.4775 | 0.4775 |
| Methylcellulose | | | |
| Carboxylic acid- | 1.75 | 2.49 | 1.0 |
| containing | | | |
| antioxidants | | | |
| Adjuvant | 0.43 | 0.43 | 0.43 |
| Water/Aqua | 39.15125 | 37.735 | 41.2505 |
| Ethyl Alcohol | 20.86 | 20.14 | 21.96 |
| | Best | worst | 2nd worst |
| | lubricity | lubricity | lubricity |
| | with high | with best | and |
| | translucency | translucency | translucency |

Compositions and formulations as described in the representative embodiments herein are selected from commercially available materials, including, for example: one of Mango Leaf Extract with from less than 50 weight percent and up to 100 weight percent mangiferin.

General Procedure for Preparation of Examples: To a glass or stainless steel beaker equipped with a stirring element and a heating element was added a portion of water and glycol, the xanthonoid, and hydrotropes and mixed until the xanthonoid was in solution with mild to moderate heating (up to about 65 degrees Celsius). To this mixture was added the carboxylic acid-containing antioxidants, and vitamin C and mixed until dissolved under 35 degrees Celsius, as well as any other adjuvants, the pantothenic acid derivatives, and hydroxypropyl methylcellulose. Once the temperature of the mixture cooled to below 35 degrees Celsius, or preferentially, below 30 degrees Celsius, the ethyl alcohol was added and the mixture was stirred until a translucent cosmetic composition was obtained. In another embodiment, the procedure above may be modified by adding all ingredients together in a beaker equipped with a heating and stirring element except the ethyl alcohol, heated to about 60 to 65 degrees Celsius for 30 min, then cooled and mixed while adding ethyl alcohol and mixing until a translucent cosmetic composition was obtained.

The objective of the instant disclosure is to generate an antioxidant serum, which has both the translucency, stability, and a satisfactory lubricity. In order to achieve performance and stability, we have shown that it is critical to control the solvent system through selection of the proper amount of glycols, including a specific amount of the hydrotropes caffeine and panthenol and formulate xanthanoid and vitamin C, which serves both to refine the solubility and translucency of the mixture as well as control the feel on skin after application, in another word the lubricity.

In the instant disclosure, it was found that a wide variety of acceptable mixtures of most ingredients were able to provide optical translucency and prevent crystallization of actives without Hydroxypropyl methylcellulose (HPMC), the lubricity and general sensory aspects of the resultant cosmetic compositions when applied on skin was not appealing. When HPMC was added to thicken the serum for sensorial reasons, we unexpectedly found that control of caffeine, panthenol, glycols, and alcohol was particularly critical for optimal translucency and stability in formulations containing high levels of vitamin C and xanthanoids. These results are seen in the above Table 1.

In another example, the specific relationship of hydrotrope, carboxylic acid-containing antioxidant, glycols, and pantothenic acid derivative was examined using a design of experiments protocol, response surface methodology. 21 Formulas were prepared and evaluated for lubricity, translucency, and stability by varying the factors as indicated in Table 2.

TABLE 2
Factors Examined in Experimental Design based on Inventive
example 1.

| Factor Name | Range Explored in Design |
|---|---|
| Hydrotrope | 0 to 2 wt. % |
| Glycols | 12 to 24 wt. % |
| Pantothenic acid derivative | 0 to 4 wt. % |
| Carboxylic acid-containing antioxidant | 0 to 4 wt. % |

The measured values of lubricity, translucency, and stability for the 21 formulas prepared based on Table 2 were used to create a response surface model for lubricity, translucency, and stability using non-linear methods. Analysis of the models allowed the inventors to understand that in the inventive example containing vitamin C and xanthonoid, there was an optimal mixture of hydrotrope (from about 0.8 wt. % to about 1.5 wt. %), glycols (from about 15 wt. % to about 24 wt. %), pantothenic acid derivative (from about 1.5 wt. % to about 2.5 wt. %), and carboxylic acid-containing antioxidant (from about 0.5 wt. % to about 2.0 wt. %, with optimal results from about 1.0 wt. % to about 2.0 wt. %).

The same experimental design using response surface methodology and factors examined in Table 2 was repeated based on a formula identical to inventive example 1, but without hydroxypropyl methylcellulose. The results of the second experimental design on 21 formulas based off inventive example 1 without hydroxypropyl methylcellulose were analyzed and the inventors found that no specific control of glycols, pantothenic acid derivative, and carboxylic acid-containing antioxidant was required for optimal lubricity, translucency, and stability; however, at least about 1.0 wt. % of hydrotrope was required for translucency.

Therefore, it was critical for the inventors to control the amounts of hydrotrope, pantothenic acid derivative, carboxylic acid-containing antioxidant, and glycols in a formula containing vitamin C, xanthonoid, and hydroxypropyl methylcellulose in order to achieve the optimal translucency, stability and lubricity.

What is claimed is:

1. A cosmetic composition comprising:
a. At least one xanthonoid;
b. At least about 10 wt. % of Vitamin C;
c. At least one hydrotrope from about 0.8 to about 1.5 wt. %.;
d. A solvent comprising water, at least one glycol and ethyl alcohol;
e. Pantothenic acid, derivatives of pantothenic acid, and mixtures thereof;
f. Hydroxypropyl methylcellulose, ethylcellulose, methyl cellulose and mixtures thereof;
g. At least one carboxylic acid-containing antioxidant; and
wherein the cosmetic composition is stable, translucent and provides a lubricated feel when applied on skin; and
wherein, all weight percentages are based on the total weight of the cosmetic composition.

2. The composition of claim 1, wherein the at least one xanthonoid is chosen from Mangifera indica leaf extract, tomentonone, zeyloxanthonone, calozeyloxanthone, morellic acid, forbesione methylswertianin, bellidifolin, mangostin, normangostin, gartanin, euxanthic acid, and mixtures thereof.

3. The composition of claim 2, wherein the at least one xanthanoid is present from about 0.1 to about 1.0 wt. % based on the total weight of the cosmetic composition.

4. The composition of claim 1, wherein the at least one hydrotrope is chosen from caffeine, hydroxy urea, nicotinic acid, derivatives of nicotinic acid, and combinations thereof.

5. The composition of claim 4, wherein the at least one hydrotrope is present from about 0.8 to about 1.2 wt. % based on the total weight of the cosmetic composition.

6. The composition of claim 1, wherein the solvent comprises water, at least one glycol chosen from pentylene glycol, propylene glycol, dipropylene glycol, and combinations thereof; and ethyl alcohol.

7. The composition of claim 1, wherein the at least one glycol is present from about 15 to about 24 wt. % based on the total weight of the cosmetic composition.

8. The composition of claim 1, wherein pantothenic acid, pantothenic acids derivatives and mixture thereof are present from about 1.5 to about 2.5 wt. % based on the total weight of the cosmetic composition.

9. The composition of claim 1, wherein pantothenic acid, pantothenic acid derivatives, or mixture thereof is panthenol.

10. The composition of claim 9, wherein the panthenol is present from about 1.5 to about 2.5 wt. % based on the total weight of the cosmetic composition.

11. The composition of claim 1, wherein Hydroxypropyl methylcellulose, ethylcellulose, methyl cellulose and mixtures thereof are present from about 0.1 to 1.5 wt. % based on the total weight of the cosmetic composition.

12. The composition of claim 11, wherein the Hydroxypropyl Methylcellulose is present from about 0.1 to 1.5 wt. % based on the total weight of the cosmetic composition.

13. A cosmetic composition comprising:
a. from about 0.1 to about 1.0% wt. % of at least one xanthonoid chosen from Mangifera indica leaf extract, tomentonone, zeyloxanthonone, calozeyloxanthone, morellic acid, forbesione methylswertianin, bellidifolin, mangostin, normangostin, gartanin, euxanthic acid, and combinations thereof;
b. from at least about 10 wt. % of Vitamin C;
c. from about 0.8 to about 1.5 wt. % of at least one hydrotrope chosen from caffeine, urea, nicotinic acid, esters, amides, or salts thereof and combinations thereof;
d. from about 18 to about 22 wt. % of a solvent comprising water and at least one glycol chosen from pentylene glycol, propylene glycol, dipropylene glycol, and combinations thereof;
e. from about 10 to about 25 wt. % of ethyl alcohol;
f. from about 1.5 to about 2.5% of Panthenol,
g. from about 0.2 to about 1.0 wt. % of Hydroxypropyl Methylcellulose;
h. from about 0.5 to about 5 wt. % of at least one carboxylic acid-containing antioxidant;
wherein the cosmetic composition is stable, translucent and provides a lubricated feel when applied to skin;
wherein all weight percentages are based on the total weight of the cosmetic composition.

14. A method for treating skin comprising applying a cosmetic composition of claim 1 to the skin.
